(19) United States
(12) Patent Application Publication
(10) Pub. No.: US 2018/0092713 A1
(43) Pub. Date: Apr. 5, 2018

(54) DENTAL TREATMENT METHOD COMBINING TOOTH POSITIONERS AND ARCH ALIGNERS

(71) Applicants: Eggert BOEHLAU, Ontario (CA); Daniel KNOCH, Hixson, TN (US)

(72) Inventors: Eggert BOEHLAU, Ontario (CA); Daniel KNOCH, Hixson, TN (US)

(21) Appl. No.: 15/284,843

(22) Filed: Oct. 4, 2016

(51) Int. Cl. A61C 7/00; A61C 7/08; A61C 9/00; A61C 19/05; A61F 5/56; A63B 71/08; B29C 67/00; B33Y 10/00; B33Y 80/00; B33Y 50/02

(52) U.S. Cl. CPC A61C 7/002; A61C 7/08; A61C 9/004; A61C 19/05; A61F 5/566; B29L 2031/753; B29C 67/0088; B33Y 10/00; B33Y 80/00; B33Y 50/02; A61F 2005/563; A63B 71/085

(57) ABSTRACT

A multiple-stage treatment method of orthodontically correcting teeth of a patient. The patient may wear, during the night and whenever convenient, a tooth positioner shaped to fit their dentition. The patient may then wear, at other times, an arch aligner shaped to fit their dentition. The patient may progress in stages, with the patient using a different arch aligner at every stage and a different tooth positioner every several stages. The combination of the arch aligner and tooth positioner may be used to treat, among other problems, class II and class III malocclusions, or transverse or vertical malocclusions, while maximizing the potential for patient compliance and reducing the number of tooth positioners and/or arch aligners that have to be made.

### BACKGROUND

The field of orthodontics generally relates to creating space within a patient's mouth, and moving the patient's teeth within that space. The most common way to accomplish this has typically been via wires and ligations (i.e. braces). However, the use of braces to readjust teeth has typically been highly labor-intensive on the part of the dentist or orthodontist, as braces often require frequent reactivations by the orthodontist to adjust the wires. Braces are also typically mounted to the teeth using an adhesive/acid system; this mounting process can be uncomfortable for some adult patients, and is often even more time-consuming for the practitioner. This also makes them fairly expensive for the patient, who will have to keep scheduling appointments with medical practitioners that typically demand a high rate of compensation.

Another reasonably common way to improve a patient's occlusion has been with the use of a tooth positioner, essentially a mold made from an impression of, or a model of an impression of, a patient's mouth. Tooth positioners merge/connect both dental arches, and provide good control how the teeth of the upper dental arch meet with the lower dental arch, and vice versa. Tooth positioners are typically constructed from an elastic material, and are in part activated by the masticatory muscles and by the elastic properties of the tooth positioner material. Tooth positioners have typically been used as a fine detailing device at the completion of conventional fixed braces orthodontic appliance therapy. Tooth positioners are capable of producing small amounts of detailed tooth movement without requiring the intercession of a dental practitioner. As such, tooth positioners are often used for the purpose of achieving final artistic positioning and retention of a patient's occlusion; for example, once a patient's occlusion is nearly ideal and further changes in wires are unlikely to solve further problems, or when a patient's interest in wearing braces has run out, an orthodontist may suggest a tooth positioner for the purpose of finishing treatment. However, such devices also suffer from downsides. Practitioners often find that long-term compliance with a tooth positioner-based treatment plan is problematic, because many patients consider the devices to be bulky and unattractive, and to interfere with oral function. As a result, it is often unrealistic to expect a patient to wear the device for the length of time needed to effect a full orthodontic treatment (which can be up to 22 hours per day).

A third common orthodontic device is an arch aligner. Arch aligners are typically thin, flat plastic aligner trays that fit over a patient's top or bottom teeth, or some portion thereof. These function similarly to tooth positioners with respect to achieving alignment within an arch, and have the advantage of being less bulky than tooth positioners. As a result, arch aligners are well tolerated by patients, as they are easy to wear, easy to remove, and result in limited interference with oral function. Patients have been shown to adapt to these appliances well for protracted periods during nighttime and even during the day. However, arch aligners are more limited in application than conventional braces or tooth positioners, and may not be able to be used for certain treatments. These treatments may include, for example, coordination of an occlusion or making anteroposterior (AP) changes, which may include, for example, class II and class III occlusal issues, transverse occlusal issues, and/or vertical occlusal issues.

### SUMMARY

According to at least one exemplary embodiment, a multiple-stage treatment method of orthodontically correcting teeth of a patient may be disclosed. First, an impression may be taken of the dentition of the patient, for example using a bite registration procedure or another modeling procedure, such as 3-D modeling. Next, using the impression of the dentition of the patient, a model of the dentition of the patient may be created, wherein the model is at least one of a digital model or a realistic model. Next, a plurality of adjustments may be made to the model, which may include adjusting a position of at least one tooth in the model; after each adjustment in the plurality of adjustments is made, an arch aligner may be created that is shaped to fit the adjusted dentition of the model. Next, after making the plurality of adjustments, a tooth positioner may be created that is shaped to fit the adjusted dentition of the model. Each of the adjustments in the plurality of adjustments may be a progressive correction of the model of the dentition of the patient from a current occlusion to an ideal occlusion. In an embodiment, the tooth positioner may also be used as a sleep apnea or bruxism treatment device. In another embodiment, a sports guard that may be used during contact sports may also be created.

According to another exemplary embodiment, a multiple-stage treatment method of orthodontically correcting an occlusion of one or more teeth may be disclosed. Such a method may include, for each stage in a plurality of stages, periodically wearing, such as during the day, until the teeth adjust from a first configuration to a second configuration, one or more arch adjusters, the one or more arch adjusters shaped to fit the one or more teeth and configured to apply a force to at least one of the one or more teeth, the at least one tooth being in a different position in the second configuration as compared to the first configuration, and the force tending to move the at least one tooth from the position of the first configuration to the position of the second configuration. The method may further include periodically wearing, such as during the night or during other times when the one or more arch aligners are not worn, until the teeth adjust from a first configuration to a second configuration, a tooth positioner, the tooth positioner shaped to fit the one or more teeth and configured to apply a force to the at least one tooth, the at least one tooth being in a different position in the second configuration as compared to the first configuration, and the force tending to move the at least one tooth from the position of the first configuration to the position of the second configuration. The method may further include, when the teeth have adjusted from the first configuration to the second configuration, exchanging the one or more arch adjusters for one or more arch adjusters shaped to fit the one or more teeth and configured to apply a force to the at least one tooth tending to move the at least one tooth from the position of the second configuration to the position of a third configuration; wherein the tooth positioner is configured to apply a force to the at least one tooth tending to move the at least one tooth from the position of the second configuration to the position of the third configuration; and wherein the first configuration and second configuration of each stage in the plurality of stages after the first are respectively the second configuration and third configuration of the previous stage. In some embodiments, at some stages, the tooth positioner may be exchanged for another tooth positioner shaped to fit the one or more teeth and configured to apply a force to the at least one tooth tending to move the at least one tooth from its current configuration to a new configuration.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

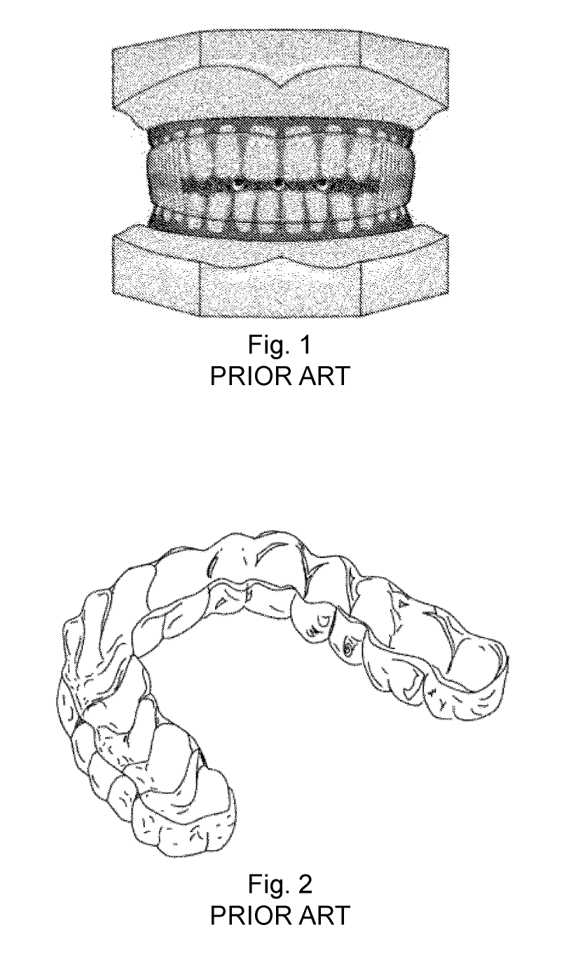

Exemplary FIG. 1 shows an exemplary embodiment of a tooth positioner that may be incorporated into an exemplary dental treatment method.

Exemplary FIG. 2 shows an exemplary embodiment of an arch aligner that may be incorporated into an exemplary dental treatment method.

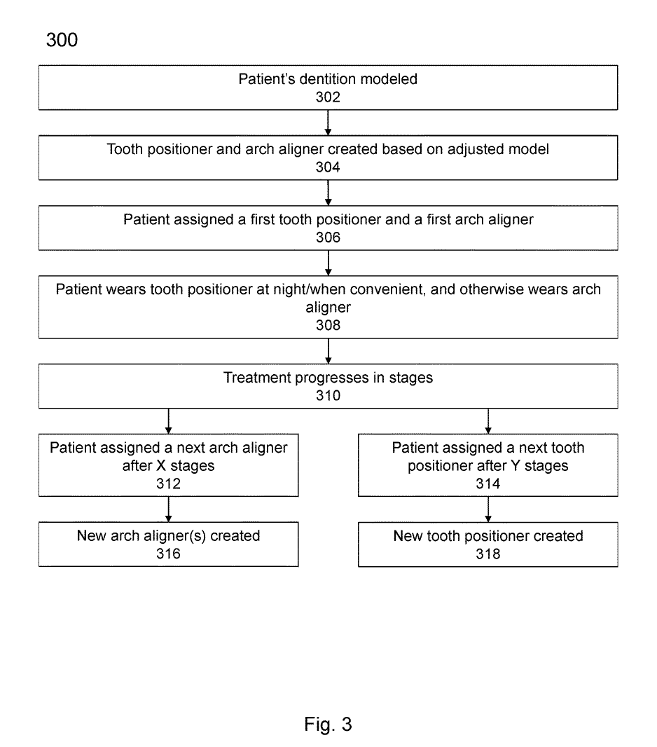

Exemplary FIG. 3 shows a process flow diagram depicting an exemplary embodiment of a treatment method.

### DETAILED DESCRIPTION OF THE EMBODIMENTS

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration.” Any embodiment described herein as “exemplary” is not necessarily to be construed as preferred or advantageous over other embodiments. Likewise, the term “embodiments of the invention” does not require that all embodiments of the invention include the discussed feature, advantage or mode of operation.

Further, many of the embodiments described herein, such as embodiments dealing with constructing digital 3D models, may be described in terms of sequences of actions to be performed by, for example, elements of a computing device. It should be recognized by those skilled in the art that the various sequences of actions described herein can be performed by specific circuits (e.g. application specific integrated circuits (ASICs)) and/or by program instructions executed by at least one processor. Additionally, the sequence of actions described herein can be embodied entirely within any form of computer-readable storage medium such that execution of the sequence of actions enables the at least one processor to perform the functionality described herein. Furthermore, the sequence of actions described herein can be embodied in a combination of hardware and software. Thus, the various aspects of the present invention may be embodied in a number of different forms, all of which have been contemplated to be within the scope of the claimed subject matter. In addition, for each of the embodiments described herein, the corresponding form of any such embodiment may be described herein as, for example, “a computer configured to” perform the described action.

According to an exemplary embodiment, and referring generally to the Figures, a dental treatment method which may involve combining one or more tooth positioners and multiple arch aligners may be disclosed. Specifically, the method may involve combined and alternating use of active individual dental arch aligner appliances, which may be applied to both dental arches or one individual arch, and active or passive tooth positioners. In an exemplary embodiment, the dental treatment method may be used to create larger tooth movements than would be possible with an arch aligner alone; for example, in an exemplary embodiment, a larger tooth movement, such as a tooth movement of around 1 mm, may be effected by the treatment method.

According to such a dental treatment method, both one or more tooth positioners and multiple arch aligners may be created for a patient. Both the tooth positioner or positioners and the arch aligners may have an occlusion geometry that differs slightly from the patient's own occlusion geometry; these differences in geometry may serve to guide the teeth of the patient to new positions.

In some exemplary embodiments, variations of tooth positioners may be used instead of or in addition to more standard tooth positioners. For example, in an exemplary embodiment, a dental appliance for treating obstructive sleep apnea may have the functionality of and may substitute for a tooth positioner. In another exemplary embodiment, a dental bruxism device may be substituted for a tooth positioner. In another exemplary embodiment, a sports guard may be used in addition to a tooth positioner and arch aligner combination; for example, in an exemplary embodiment, a patient may be directed to wear the sports guard when playing sports or contact sports, may be directed to wear a tooth positioner when convenient (such as during sleep or the like), and may be directed to wear an arch aligner at all other times.

Turning now to exemplary FIG. 1, an exemplary embodiment of a tooth positioner that may be incorporated into an exemplary dental treatment method may be disclosed. Tooth positioners, such as the one disclosed in FIG. 1, may generally be known in the prior art, though variant tooth positioners may exist. For example, according to an exemplary embodiment, a tooth positioner may be formed from a single piece and may be fitted such that it fits over both the patient's upper and lower arches. According to another exemplary embodiment, the tooth positioner may be formed from multiple pieces connected by a hinge. Other tooth positioner variants may also be used. In an exemplary tooth positioner, both the upper arch and the lower arch of the tooth positioner may be fitted to the actual or desired geometry of the patient's teeth. Tooth positioner may also optionally include a number of holes for airway management.

Turning now to exemplary FIG. 2, an exemplary embodiment of an arch aligner that may be incorporated into an exemplary dental treatment method may be disclosed. Arch aligners, such as the one disclosed in FIG. 2, may generally be known in the prior art, though variant arch aligners may exist. For example, according to an exemplary embodiment, an arch aligner may be a shallow tray formed from plastic and having depressions and raised surfaces that may reflect a patient's full dentition and which may be adhered to the patient's teeth. Other arch aligner variants may include, for example, an arch aligner that may reflect only a portion of a patient's dentition and which may be adhered to those teeth in particular.

Turning now to exemplary FIG. 3, an exemplary embodiment of a treatment method 300 may be disclosed. According to an exemplary embodiment of the treatment method 300, a patient's dentition first may be modeled 302. For example, according to an exemplary embodiment, a bite registration procedure may be used to generate an impression that can be used to construct a model of a patient's dentition. In another exemplary embodiment, a model of a patient's dentition may be constructed by using a 3-D scanner to scan the patient's dentition directly, and then using dental or orthodontic modeling software to construct a digital model of the patient's dentition. This may include, for example, modeling the way that the patient's dental arches relate to each other (the patient's dental articulation or gliding occlusion). The dental or orthodontic modeling software may then be used to plan tooth movements, desired changes to dental articulation/gliding occlusion, and other orthodontic treatment goals, as desired. According to another exemplary embodiment, another method or combination of methods of generating a model may be used instead, as desired. Model may be digital or realistic, or a combination of the two, as desired.

One or more tooth positioners and arch aligners may then be created based on the model 304. Tooth positioners and arch aligners may be created using any method known in the art. For example, according to an exemplary embodiment, tooth positioners may be 3-D printable. For example, according to an exemplary embodiment, tooth positioners may be constructed from a 3-D printable material that is soft and is bio-compatible. Other methods of constructing either the one or more tooth positioners or the one or more arch aligners may be used instead.

According to an exemplary embodiment, the patient may be assigned at least a first tooth positioner and a first arch aligner 306; however, according to other exemplary embodiments, the patient may additionally be assigned additional arch aligners, or additional tooth positioners, as desired. For example, according to an exemplary embodiment, multiple sets of arch aligners may be formed for every tooth positioner that is to be created. The patient may be instructed to wear a tooth positioner at opportune times, such as at night or whenever may be convenient during the day. The patient may then be instructed to, during the day or whenever else it might be inconvenient to them to wear the tooth positioner, wear one of the arch aligners 308. During the evening, or whenever the tooth positioner is worn, the tooth positioner may act to readjust or retain the patient's occlusion, and may guide the teeth into place or may prevent the teeth from shifting back to old positions. During the day, or whenever the arch aligners are worn, the arch aligners may act to readjust or retain the patient's top and/or bottom arches, without addressing any occlusal issues that the patient may have. According to an exemplary embodiment, the combination of the tooth positioner and arch aligner may maximize the patient's exposure to the tooth positioner while minimizing the inconvenience that the patient has to endure to make use of the tooth positioner, which may ensure that the patient does not feel inconvenienced by the treatment method, and may also more reliably ensure that the patient completes the treatment regimen.

According to an exemplary embodiment, a patient's treatment may take place in stages 310. In such an embodiment, a patient may be given one or more tooth positioners and one or more arch aligners, with a first tooth positioner to be worn during the evening and a first arch aligner to be worn during the day. According to such an embodiment, the first tooth positioner of the patient may be exchanged for a second tooth positioner of the patient 314 after the patient has progressed through a set number of stages, such as four or five stages, of the treatment method. However, the patient may exchange their arch aligner 312 more often than they exchange the tooth positioner 314; for example, they may exchange their arch aligner 312 for a new arch aligner at, for example, every stage, or after a lower set number of stages, such as after every two stages.

According to an exemplary embodiment, the geometry of the arch aligners, as well as the geometry of the tooth positioners, may progressively vary. For example, according to an exemplary embodiment, the geometry of the arch aligner that a patient uses at a first stage 306 (“first arch aligner”) may be similar to the patient's existing occlusion, but may have minor differences in spacing so as to assist in aligning or retaining the patient's teeth in a first intermediate alignment, wherein the first intermediate alignment is an intermediate-stage occlusion between the original occlusion of the patient's dentition and the desired occlusion found in the tooth positioner that the patient is using in the first stage 306 (“first tooth positioner”). According to an exemplary embodiment, once the patient's occlusion has reached this first intermediate alignment, the patient may switch from using a first arch aligner during the day to using a second arch aligner during the day 312, while still continuing to use the same tooth positioner that they have been using—the first tooth positioner mentioned previously—at night. The geometry of a second arch aligner may be less similar than the geometry of the first arch aligner to the patient's existing occlusion, having even more minor differences in spacing so as to assist in aligning or retaining the patient's teeth in a second intermediate alignment, and may be more similar to the geometry of the first tooth positioner. According to an exemplary embodiment, the patient may then continue this treatment, and may, for example, exchange a second arch aligner for a third, or a third arch aligner for a fourth, and so on and so forth, once they reach an appropriate stage of the treatment.

According to an exemplary embodiment, an arch aligner other than the first arch aligner, such as the fourth or fifth arch aligner in a series or another arch aligner in the series, may have an arch geometry identical or substantially identical to the equivalent arch geometry of the first tooth positioner. For example, if the arch aligner in question is intended to be worn on the top arch, then it may be identical to the top arch geometry of the tooth positioner. Once the patient has begun using such an arch aligner, and has used the arch aligner for an appropriate period of time, the patient may need to switch to a new tooth positioner as well as a new arch aligner. As such, according to an exemplary embodiment, in the next stage in the patient's treatment, the patient may exchange both their tooth positioner and their arch aligner for appliances having different geometries. The new tooth positioner of the patient, the second tooth positioner, may have different geometry from the first tooth positioner; the geometry of this second tooth positioner may be closer to a desired occlusion for the patient. According to an exemplary embodiment, wherein the patient is assigned a next arch aligner after every X stages 312, and wherein the patient is assigned a next tooth positioner after every Y stages 314, the geometry of the arch aligner and the equivalent geometry of the tooth positioner may be identical at every stage having a number evenly divisible by either X or Y. According to another exemplary embodiment, tooth positioners and arch aligners may have different internal geometry, and tooth positioners and arch aligners may be exchanged at different times.

According to one exemplary embodiment, the geometries of the tooth positioners and the arch aligners may be based on either the actual progression of the patient's occlusion, and the patient may be regularly fitted for new arch aligners and/or tooth positioners, as desired. According to another exemplary embodiment, the geometries of the arch aligners and/or the tooth positioners may be based on the anticipated progression of the patient's occlusion, allowing a number of them to be generated based on one data set collected from the patient. According to another exemplary embodiment, a combination of both actual and anticipated progression may be used, as desired. According to an exemplary embodiment wherein the actual progression of the patient is used, a new arch aligner 316 or set of arch aligners, or a new tooth positioner 318, may be created after every stage or set of stages that the patient progresses through. For example, according to an exemplary embodiment, the patient's new occlusion may be measured after every five stages of the treatment, and five new arch aligners 316 and one new tooth positioner 318 may be created for the patient to use during the next five stages of the treatment. Alternatively, the patient's occlusion may be measured at different times, or arch aligners 316 or tooth positioners 318 may be created at different times, if desired.

To give an example of how the method may be implemented, in an exemplary embodiment, a patient's treatment may have twenty total stages 310. The patient may exchange an arch aligner 312 for a new arch aligner at every one of the twenty different stages. The patient may then exchange a tooth positioner 314 at every fifth stage of the treatment, at which point the patient would then progress to the next tooth positioner. This means that a total of twenty different arch aligners would be created 316 for the patient, and four different tooth positioners 318 would be created for the patient, with the patient exchanging tooth positioners at the fifth, tenth, and fifteenth stages of the treatment. However, in other exemplary embodiments, a treatment may be created to have a different number of total stages 310 or a different number of stages between arch aligners 312 or tooth positioners 314.

According to an exemplary embodiment, the above treatment method may be used to treat a variety of conditions. For example, according to an exemplary embodiment, the above treatment method may be used to treat class II or class III malocclusions. In another exemplary embodiment, the above treatment method may be used to make transverse or vertical occlusal changes. According to another exemplary embodiment, the above treatment method may be used to otherwise coordinate an occlusion or make anteroposterior changes, or may be used to treat other conditions, as desired.

Other embodiments other than those set forth above may also be contemplated. For example, in an exemplary embodiment, instead of creating multiple tooth positioners 318 for a particular treatment regimen, one or a smaller number of modular tooth positioners, each of which may be adapted to hold an arch aligner or a plurality of arch aligners corresponding to a patient's top and/or bottom teeth, may be created. In such an embodiment, a patient may properly configure the tooth positioner by inserting the appropriate arch aligner into a top or bottom section of the tooth positioner, or by inserting the tooth positioner over an arch aligner that they are wearing, and may then wear the tooth positioner as they otherwise would 308. This may reduce the number of items that may be custom-printed for a patient, if desired.

The foregoing description and accompanying drawings illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. A multiple-stage treatment method of orthodontically correcting teeth of a patient, comprising:
taking an impression of the dentition of the patient;
creating, using the impression of the dentition of the patient, a model of the dentition of the patient, wherein the model is at least one of a digital model or a realistic model;
making a plurality of adjustments to the model comprising adjusting a position of at least one tooth in the model, and after each adjustment in the plurality of adjustments, creating an arch aligner shaped to fit the adjusted dentition of the model; and
after making the plurality of adjustments, creating a tooth positioner shaped to connect both an upper dental arch and a lower dental arch and to fit the adjusted dentition of the model,
wherein an internal geometry of the arch aligner is different from an internal geometry of the tooth positioner for each adjustment in the plurality of adjustments, and
wherein each of the adjustments in the plurality of adjustments is a progressive correction of the model of the dentition of the patient from a current occlusion to an ideal occlusion.

2. The method of claim 1, wherein the step of taking an impression of the dentition of the patient comprises at least one of a bite registration procedure and 3-D scanning.

3. The method of claim 1, wherein the step of creating a tooth positioner shaped to fit the adjusted dentition of the model comprises printing, using a 3D printer, a tooth positioner; and
wherein the tooth positioner is constructed from a soft and biocompatible material.

4. The method of claim 1, wherein the tooth positioner functions as at least one of a sleep apnea treatment device or a bruxism treatment device.

5. The method of claim 1, wherein the method further comprises creating a sports guard appliance shaped to fit the adjusted dentition of the model.

6. A multiple-stage treatment method of orthodontically correcting an occlusion of one or more teeth, comprising:
for each stage in a plurality of stages:
periodically wearing, until the teeth adjust from a first configuration to a second configuration, one or more arch adjusters, the one or more arch adjusters shaped to fit the one or more teeth and configured to apply a force to at least one of the one or more teeth, the at least one tooth being in a different position in the second configuration as compared to the first configuration, and the force tending to move the at least one tooth from the position of the first configuration to the position of the second configuration;
periodically wearing, until the teeth adjust from a first configuration to a second configuration, a tooth positioner, the tooth positioner shaped to connect both an upper dental arch and a lower dental arch and to fit the one or more teeth and configured to apply a force to the at least one tooth, the at least one tooth being in a different position in the second configuration as compared to the first configuration, and the force tending to move the at least one tooth from the position of the first configuration to the position of the second configuration; and
when the teeth have adjusted from the first configuration to the second configuration, exchanging the one or more arch adjusters for one or more arch adjusters shaped to fit the one or more teeth and configured to apply a force to the at least one tooth tending to move the at least one tooth from the position of the second configuration to the position of a third configuration,
wherein an internal geometry of the arch aligner is different from an internal geometry of the tooth positioner for each stage in the plurality of stages,
wherein the tooth positioner is configured to apply a force to the at least one tooth tending to move the at least one tooth from the position of the second configuration to the position of the third configuration, and
wherein, for each stage in the plurality of stages after the first, the first configuration of the stage is the second configuration of the previous stage, and the second configuration of the stage is the third configuration of the previous stage.

7. The method of claim 6, wherein the step of periodically wearing one or more arch adjusters is performed during the day, and wherein the step of periodically wearing the tooth positioner is performed during the night.

8. The method of claim 6, wherein, after each of the stages in the plurality of stages have been completed, the tooth positioner is exchanged for a second tooth positioner shaped to fit the one or more teeth and configured to apply a force to the at least one tooth tending to move the at least one tooth from the position of a current configuration of the at least one tooth to a position of a new configuration of the at least one tooth.

9. The method of claim 6, further comprising wearing, during performance of a sporting activity, a sports guard, the sports guard shaped to fit the one or more teeth and configured to apply a force to the at least one tooth, the at least one tooth being in a different position in the second configuration as compared to the first configuration, and the force tending to move the at least one tooth from the position of the first configuration to the position of the second configuration.

10. The method of claim 9, further comprising:
when the teeth have adjusted from the first configuration to the second configuration, exchanging the sports guard for a sports guard shaped to fit the one or more teeth and configured to apply a force to the at least one tooth tending to move the at least one tooth from the position of the second configuration to the position of the third configuration.

11. The method of claim 6, wherein the tooth positioner functions as at least one of a sleep apnea treatment device or a bruxism treatment device.

12. The method of claim 6, wherein the tooth positioner is constructed from a soft and biocompatible material, and wherein the tooth positioner is formed via 3-D printing.

13. The method of claim 6, wherein the method is used to correct at least one of: a class II malocclusion, a class III malocclusion, a transverse malocclusion, or a vertical malocclusion.

14. The method of claim 6, wherein the plurality of stages is one of: four stages in length or five stages in length.

15. The method of claim 8, wherein the tooth positioner and the second tooth positioner are worn for the same number of stages.

16. The method of claim 8, wherein the difference between the current configuration of the at least one tooth and the new configuration of the at least one tooth is less than one millimeter.
